(19) United States
(12) Patent Application Publication
(10) Pub. No.: US 2010/0303861 A1
(43) Pub. Date: Dec. 2, 2010

(54) Live Attenuated Vaccine Strain for Prevention of Tularemia

(75) Inventors: Arthur O. Tzianabos, Reading, MA (US); Dennis L. Kasper, Charlestown, MA (US); Shite Sebastian, Malden, MA (US); Eric Rubin, Waban, MA (US); Simon T. Dillon, Danvers, MA (US)

Correspondence Address: Harvard University & Medical School;c/o Wolf, Greenfield & Sacks, P.C., 600 Atlantic Avenue, Boston, MA, 02210-2206, US

(73) Assignee: President and Fellows of Harvard College, Cambridge, MA (US)

(21) Appl. No.: 11/992,429

(22) PCT Filed: Sep. 22, 2006
(86) PCT No.: PCT/US06/36910

§ 371 Date: Jul. 12, 2010

Related U.S. Application Data
(60) Provisional application No. 60/720,004, filed on Sep. 24, 2005.

(51) Int. Cl. A61K 39/02; A61P 31/04; C12N 15/74; C12Q 1/02; G01N 33/53; C12Q 1/68

(52) U.S. Cl. 424/234.1; 435/471; 435/29; 435/7.6; 435/6

(57) ABSTRACT

The invention provides live attenuated avirulent strains of Francisella tularensis, as well as methods for their preparation and use in protecting a mammal against infection with F. tularensis and against tularemia.

### BACKGROUND OF THE INVENTION

Francisella tularensis (F. tularensis) is a pleomorphic Gram-negative facultative intracellular pathogen that is the etiological agent of the potentially fatal human disease, tularemia. Ellis J et al. (2002) Clin Microbiol Rev 15:631-46. The high virulence, low infectious dose, and aerosolized nature of transmission of F. tularensis have raised serious concerns for the exploitation of this microbe as a biowarfare agent. Dennis D T et al. (2001) JAMA 285:2763-73. Two major biovars of F. tularensis exist, namely, F. tularensis subsp. tularensis (type A) and F. tularensis subsp. holarctica (type B), both of which remain highly virulent and infectious against a wide range of mammalian species including humans. Ellis J et al. (2002) supra. An empirically derived vaccine strain of F. tularensis referred to as Live Vaccine Strain (LVS; F. tularensis LVS; Ft. LVS) exists and is currently being administered tout risk individuals. Sandstrom G (1994) J Chem Technol Biotechnol 59:315-20. However, the lack of complete understanding for the attenuation of the strain, unwanted side-effects, incomplete immunity to the vaccines, and the scarcity of information regarding the virulence factors of this bacterium have hindered the licensing of this strain to be used as a generalized vaccine in the United States. Sjostedt A (2003) Curr Opin Microbiol 6:66-71; Burke D S (1977) J Infect Dis 135:55-60.

Although limited knowledge exists regarding the virulence factors of this microorganism, the surface polysaccharides, namely, the lipopolysaccharide (LPS) and the capsular polysaccharide, have emerged as virulence determinants of this infectious pathogen. Ellis J et al. (2002) supra; Sandstrom G et al. (1988) Infect Immun 56:1194-202; Cherwonogrodzky J W et al. (1994) Vaccine 12:773-5. The LPS of F. tularensis, similar to other microorganisms, consists of lipid A, core oligosaccharide, and O antigen polysaccharide. However, unlike other pathogenic bacteria, the LPS of F. tularensis is atypical in that it does not invoke a proinflammatory cytokine response and is considered to be non-toxic. Sandstrom G et al. (1992) FEMS Microbiol Immunol 5:201-10. This low level of endotoxicity has been attributed to unusual structural features of the lipid A, which lacks phosphate substituents and has been reported to be tetraacetylated. Vinogradov E et al. (2002) Eur J Biochem 269:6112-8.

The O-antigen structures from both type A and type B strains of F. tularensis have been elucidated and demonstrated to have identical tetrasaccharide repeats of the rare sugars 2-acetamido-2,6-dideoxy-D-glucose (D-QuiNAc), 4,6-dideoxy-4-formamido-D-glucose (D-Qui4NFm), and 2-acetamido-2-deoxy-D-galacturonamide (D-GalNAcAN). Vinogradov E V et al. (1991) Carbohydr Res 214:289-97; Conlan J W et al. (2002) Vaccine 20:3465-71.

Interestingly, the LPS in F. tularensis LVS has been reported to be under phase variation via an unknown mechanism (Cowley S C et al. (1996) Mol Microbiol 20:867-74), and the state of LPS is thought to dictate the survival of F. tularensis LVS in macrophages. The resulting LPS variant (Fn. LPS type) has both an antigenically distinct O antigen polysaccharide and a functionally different lipid A moiety that resembles the LPS of the closely related subspecies Francisella novicida. Cowley S C et al. (1996) supra. The inert state of LPS (Ft. LPS type) fails to stimulate nitric oxide (NO) production from the resident macrophage and thus provides an ideal niche protected from the host immune system. In contrast, the altered phase variant state of LPS (Fn. LPS type) stimulates proinflammatory cytokines and enhanced NO production, resulting in the efficient killing of this intracellular pathogen. Cowley S C et al. (1996) supra. Thus the ability of F. tularensis to fine tune the levels of NO to bacteriostatic levels via the phase variation of its LPS is thought to be key for prolonged survival and the successful establishment of infection by this microbe.

Unlike the LPS of F. tularensis, the capsular polysaccharide from this organism has not been well studied. The encapsulation of F. tularensis is supported by earlier electron microscopy studies which demonstrated the presence of an outer layer surrounding the pathogen. Cherwonogrodzky J W et al. (1994) supra; Geisbert T W et al. (1993) J Clin Microbiol 31:1936-9. These studies further demonstrated an increased encapsulation (via growth on Chamberlain's synthetic medium at pH 6.5) of the LVS strain, resulting in this strain's approximately 1000-fold increase in virulence in mice. Cherwonogrodzky J W et al. (1994) supra. In addition, a genetically uncharacterized, acridine orange-derived Cap⁻ mutant of F. tularensis LVS has been isolated and shown to be devoid of the extracellular layer. Sandstrom G et al. (1988) supra. The capsule of F. tularensis is proposed to be key in conferring resistance to the bactericidal action of nonimmune human serum, thus facilitating survival during the bacteremic phase of this infectious pathogen. Sandstrom G et al. (1988) supra; Sorokin V M et al. (1996) FEMS Immunol Med Microbiol 13:249-52.

Although the participation of LPS and capsule in the virulence of F. tularensis has been established, the genetic loci coding for these exopolysaccharides have not been well elucidated. The only indication to date regarding the genetic loci responsible for the synthesis of the complex sugars that constitute the exopolysaccharides has been inferred from in silico analysis of the recently completed sequence of the F. tularensis subsp. tularensis Schu S4 (type A) genome. Larsson P et al. (2005) Nat Genet 37:153-9; Prior J L et al. (2003) J Med Microbiol 52:845-51. This analysis has identified a 17 kilobase (kb) gene cluster which, based on homology studies, is predicted to encode for the enzymes responsible for the biosynthesis of the O antigen polysaccharide repeating unit of this pathogen. This locus contains fifteen tightly linked genes, all transcribed in the same direction and likely organized as an operon. The putative functions of the gene products in this locus are in good agreement with the enzymes necessary for the biosynthesis of the O antigen polysaccharide repeat unit as determined for F. tularensis. Prior J L et al. (2003) supra. Homologs of Bacillus anthracis capB and capC have also been identified in the F. tularensis genome, raising the possibility of a B. anthracis polyglutamate-like capsule in this species. Larsson P et al. (2005) supra. Furthermore, a 13 kb polysaccharide locus has also been identified in the F. tularensis genome with potential to code for surface polysaccharide. Ibid. Although these regions have been proposed to be involved in exopolysaccharide biosynthesis, without mutational or phenotypic analyses, it is often not possible to determine if a polysaccharide biosynthesis locus encodes an O-antigen polysaccharide or a capsular polysaccharide.

### SUMMARY OF THE INVENTION

The invention relates to vaccines, methods of making vaccines, methods of treatment, methods of screening for therapeutics useful in treating infectious disease, and attenuated strains of Francisella tularensis. The invention involves the discovery that genetic mutations affecting the locus involved in the biosynthesis of the O antigen can yield attenuated avirulent strains of Francisella tularensis which are immunogenic and protective against infection.

In experiments described below, a transposon was used to mutangenize the chromosome of F. tularensis LVS and generate mutants of this pathogen characterized by drastically reduced expression of wild-type O antigen polysaccharide. A strain so produced was devoid of the characteristic laddering by O-antigen polysaccharide. The strain was demonstrated to be impaired in its ability to disseminate to reticuloendothelial organs and remained avirulent when tested in a murine model of tularemia.

According to one aspect of the invention, a vaccine composition is provided. The vaccine includes an effective amount of a live attenuated avirulent strain of Francisella tularensis, wherein the live attenuated strain has reduced expression of wild-type O antigen as compared to pathogenic Francisella tularensis. The vaccine also includes a pharmaceutically acceptable carrier. In one embodiment, the reduced expression results from a mutation of a gene affecting O-antigen biosynthesis. In this embodiment, the mutation can be a mutation outside of but affecting an O-antigen biosynthesis locus or it can be a mutation within and affecting an O-antigen biosynthesis locus. In one embodiment, the mutation is to a gene within an O-antigen biosynthesis locus. In one important embodiment, the mutation is a mutation of wbtA. In another important embodiment, the strain is an avirulent strain of F. tularensis LVS. In any of the foregoing embodiments, the reduced expression of wild-type O antigen can be no expression of wild-type O antigen. In any of the foregoing embodiments, the mutation can result in the absence of characteristic laddering by O-antigen polysaccharide. In some embodiments, the effective amount is at least 10⁴ colony forming units. In one embodiment, the effective amount can be 10⁴ to 10⁸ colony forming units.

According to another aspect of the invention, a method is provided for vaccinating a mammal against infection by a pathogenic strain of Francisella tularensis. The method involves administering to the mammal an effective amount of a live attenuated avirulent strain of Francisella tularensis, wherein the live attenuated strain has reduced expression of wild-type O antigen as compared to pathogenic Francisella tularensis. In one embodiment, the reduced expression results from a mutation of a gene affecting O-antigen biosynthesis. In this embodiment, the mutation can be a mutation outside of but affecting an O-antigen biosynthesis locus or it can be a mutation within and affecting an O-antigen biosynthesis locus. In one embodiment, the mutation is to a gene within an O-antigen biosynthesis locus. In one important embodiment, the mutation is a mutation of wbtA. In another important embodiment, the strain is an avirulent strain of F. tularensis LVS. In any of the foregoing embodiments, the reduced expression of wild-type O antigen can be no expression of wild-type O antigen. In any of the foregoing embodiments, the mutation can result in the absence of characteristic laddering by O-antigen polysaccharide. In some embodiments, the effective amount is at least 10⁴ colony forming units. In one embodiment, the effective amount can be 10⁴ to 10⁸ colony forming units. The administration can be by any conventional means, as described in greater detail below. In one embodiment, the administration is intranasal. In another embodiment, the administration is intradermal. In important embodiments, the mammal is a human.

According to another aspect of the invention, a method is provided for generating antibodies or cytotoxic lymphocytes to a pathogenic strain of Francisella tularensis. The method involves administering to a mammal an effective amount of a live attenuated avirulent strain of Francisella tularensis, wherein the live attenuated strain has reduced expression of wild-type O antigen as compared to pathogenic Francisella tularensis. In one embodiment, the reduced expression results from a mutation of a gene affecting O-antigen biosynthesis. In this embodiment, the mutation can be a mutation outside of but affecting an O-antigen biosynthesis locus or it can be a mutation within and affecting an O-antigen biosynthesis locus. In one embodiment, the mutation is to a gene within an O-antigen biosynthesis locus. In one important embodiment, the mutation is a mutation of wbtA. In another important embodiment, the strain is an avirulent strain of F. tularensis LVS. In any of the foregoing embodiments, the reduced expression of wild-type O antigen can be no expression of wild-type O antigen. In any of the foregoing embodiments, the mutation can result in the absence of characteristic laddering by O antigen polysaccharide. In some embodiments, the effective amount is at least 10⁴ colony forming units. In one embodiment, the effective amount can be 10⁴ to 10⁸ colony forming units. The administration can be by any conventional means, as described in greater detail below. In one embodiment, the administration is intranasal. In another embodiment, the administration is intradermal. The antibodies and/or cytotoxic lymphocytes can be identified and isolated by any conventional means well known to those of ordinary skill in the art.

According to another aspect of the invention, a method is provided for making a live attenuated avirulent strain of Francisella tularensis. The invention involves introducing a mutation into a gene within an O-antigen biosynthesis locus of a pathogenic strain of Francisella tularensis, wherein the mutation results in reduced expression of wild-type O antigen by the pathogenic strain of Francisella tularensis. In one important embodiment, the mutation is to wbtA. In another important embodiment, the live attenuated avirulent strain is an avirulent strain of F. tularensis LVS. In any of the foregoing embodiments, the reduced expression of wild-type O antigen can be no expression of wild-type O antigen. In any of the foregoing embodiments, the mutation can result in the absence of characteristic laddering by O-antigen polysaccharide. In important embodiments, the pathogenic strain of Francisella tularensis is Francisella tularensis type A.

According to another aspect of the invention, live attenuated avirulent strains of Francisella tularensis are provided. In some embodiments, the strains have a mutation to a gene within an O-antigen biosynthesis locus of a pathogenic strain of Francisella tularensis, wherein the mutation results in reduced expression of wild-type O antigen by the pathogenic strain of Francisella tularensis. In one important embodiment, the mutation is to wbtA. In another important embodiment, the live attenuated avirulent strain is an avirulent strain of F. tularensis LVS. In any of the foregoing embodiments, the reduced expression of wild-type O antigen can be no expression of wild-type O antigen. In any of the foregoing embodiments, the mutation can result in the absence of characteristic laddering by O-antigen polysaccharide. In important embodiments, the pathogenic strain of Francisella tularensis is Francisella tularensis type A.

According to another aspect of the invention, a method is provided for identifying a candidate vaccine of a live attenuated strain of Francisella tularensis. The method involves comparing expression of wild-type O antigen by a mutant strain of Francisella tularensis to expression of wild-type O antigen by a pathogenic strain of Francisella tularensis; and identifying the mutant strain of Francisella tularensis as a candidate vaccine when the mutant strain has reduced expression of wild-type O antigen compared to the pathogenic strain. In one embodiment, the reduced expression results from a mutation of a gene affecting O-antigen biosynthesis. In this embodiment, the mutation can be a mutation outside of but affecting an O-antigen biosynthesis locus or it can be a mutation within and affecting an O-antigen biosynthesis locus. In one embodiment, a mutation is made to a gene within an O-antigen biosynthesis locus. In one important embodiment, the mutation is a mutation of wbtA. In another important embodiment, the strain is an avirulent strain of F. tularensis LVS. In any of the foregoing embodiments, the reduced expression of wild-type O antigen can be no expression of wild-type O antigen. In any of the foregoing embodiments, the mutation can result in the absence of characteristic laddering by O-antigen polysaccharide.

According to another aspect of the invention, a method is provided for identifying an agent that binds to and inhibits activity of an enzyme involved in biosynthesis of wild-type O antigen of a pathogenic strain of Francisella tularensis. The method involves contacting an enzyme involved in biosynthesis of wild-type O antigen of a pathogenic strain of Francisella tularensis with a test agent; measuring activity of the enzyme in presence of the test agent; and determining whether the test agent binds to and inhibits activity of the enzyme. In one embodiment, the enzyme involved in biosynthesis of wild-type O antigen of the pathogenic strain of Francisella tularensis is WbtA.

According to another aspect of the invention, a method is provided for identifying an agent that binds to and inhibits activity of a gene involved in biosynthesis of wild-type O antigen of a pathogenic strain of Francisella tularensis. The method involves contacting a gene involved in biosynthesis of wild-type O antigen of a pathogenic strain of Francisella tularensis with a test agent; measuring activity of the gene in presence of the test agent; and determining whether the test agent binds to and inhibits activity of the gene. In one embodiment, the gene involved in biosynthesis of wild-type O antigen of the pathogenic strain of Francisella tularensis is wbtA.

These and other aspects of the invention are described further below.

### BRIEF DESCRIPTION OF THE DRAWINGS

The figures are illustrative only and are not required for enablement of the invention disclosed herein.

FIG. 1 summarizes mutagenesis of F. tularensis live vaccine strain (LVS). FIG. 1A is a schematic representation of mariner-based mutagenesis of F. tularensis LVS. The suicide delivery plasmid pSD was utilized to introduce the Himar1 transposon into Francisella (left panel). The transposon is represented with inverted flanking repeats containing the Tn903-derived kanamycin open reading frame (ORF). The C9 transposase (C9Tpase) encoded by the suicide delivery plasmid facilitates the random transposition of the Himar1 transposon into the F. tularensis LVS chromosome (right panel). FIG. 1B is an image of a DNA gel showing Southern hybridization analysis of F. tularensis LVS wild-type and insertion mutants, as indicated on the top of the panel. FIG. 1C is a graph showing in vitro growth kinetics of F. tularensis LVS wild-type and mutant strains. The x-axis represents time in hours and the y-axis represents optical density (OD) at 620 nm.

FIG. 2 is a series of electron micrograph images demonstrating attachment of the protein A-conjugated immunogold on the surface of F. tularensis wild-type and mutant strains following incubation with the O-antigen monoclonal antibody MAb 2033. FIG. 2A shows F. tularensis wild-type. FIG. 2B shows Ft. LVSΩcapB. FIG. 2C shows Ft. LVSΩwbtA.

FIG. 3 is a series of images of western immunoblot characterization of F. tularensis LVS wild-type, wbtA and capB mutants. FIG. 3A is images of immunoblots probed at a final dilution of 1:3000 with anti-FT serum, (1); MAb 2033, (2); and MAb 2034, (3). FIG. 3B shows immunoblot analysis with adsorbed anti-FT serum. Samples were probed with anti-FT serum adsorbed with Ft. LVS wild-type strain (1); Ft. LVSΩcapB strain (2) and Ft. LVSΩwbtA strain (3). Lanes (all panels, left to right): protein standard marker; Ft. LVS; Ft. LVSΩwbtA; and Ft. LVSΩcapB.

FIG. 4 is two graphs showing F. tularensis LVS colonization studies. The colonization status of the spleen, kidney, lung, and liver tissues of male BALB/c mice was assessed four days post-challenge with the F. tularensis wild type and Ft. LVSΩwbtA strains. The bacterial load in each of the organs is represented as cfu/gram on a logarithmic scale. n=5 was used unless otherwise stated. FIG. 4A summarizes data from intradermal administration. FIG. 4B summarizes data from intranasal administration.

FIG. 5 is a graph depicting survival, following challenge with fully virulent FSC type B F. tularensis, of naïve mice (No treatment (squares)), mice previously immunized with Ft. LVS (triangles), and mice previously immunized with Ft. LVSΩwbtA (circles).

### DETAILED DESCRIPTION OF THE INVENTION

The invention provides compositions and methods relating to live attenuated avirulent strains of F. tularensis, the pathogen of tularemia. The live attenuated strains of F. tularensis of the invention are avirulent but immunogenic, i.e., suitable for use in a vaccine. As described in detail below, the live attenuated strains of F. tularensis of the invention are characterized by their reduced expression of wild-type O-antigen polysaccharide as compared to pathogenic F. tularensis. In one embodiment the reduced expression is essentially a complete lack of expression.

F. tularensis is a Gram-negative coccobacillus that causes the disease tularemia. The genus Francisella includes F. tularensis, F. novicida, and F. philomiragia. F. tularensis has at least three subspecies, including tularensis, holarctica, and mediaasiatica. Strains of F. tularensis subsp. tularensis (also termed type A) are highly virulent in humans, while F. tularensis subsp. holarctica (also termed type B) and F. tularensis subsp. mediaasiatica are associated with milder forms of tularemia. Strains of F. tularensis subsp. tularensis specifically include but are not limited to strains Schu S4, 199, and 041. Strains of F. tularensis subsp. holarctica specifically include but are not limited to LVS, 200, 025, 075, and HN63.

Pathogenic strains of F. tularensis are characterized by their virulence, i.e., their ability to infect a host, evade or overcome natural host defenses, and thus cause symptoms of the disease tularemia. Virulence depends in part on the ability of the microorganism to evade detection and elimination by elements of the immune system of an infected host, and such ability in turn can depend on the amount or structure of certain virulence factors, i.e., molecules expressed by the microorganism. As used herein, a pathogenic strain of F. tularensis can include but is not limited to those listed above. In one embodiment the pathogenic strain of F. tularensis is a strain of F. tularensis subsp. tularensis (also termed type A). In one embodiment the pathogenic strain of F. tularensis is F. tularensis subsp. holarctica LVS (hereinafter F. tularensis LVS and, equivalently, Ft. LVS).

Larsson et al. recently reported a complete genome sequence of F. tularensis subsp. tularensis Schu S4, a highly virulent isolate of F. tularensis (1,892,819 bp; GenBank accession no. AJ749949, the entire contents of which are incorporated by reference herein). See also Larsson P et al. (2005) Nat Genet 37:153-9.

A strain that is live is one that can reproduce when placed in suitable conditions. Such strain can include a strain that is actively dividing in vivo or in vitro, as well as a strain that is suspended in its growth, e.g., by lyophilization or freezing. An example of a live attenuated strain of F. tularensis is F. tularensis LVS. This strain has been described and is currently in limited use as a vaccine strain, although it is not avirulent. Sandstrom G (1994) supra. As noted above, this strain has been reported to undergo phase variation of its LPS, whereby in one state it expresses a form of LPS that stimulates proinflammatory cytokines and enhanced NO production. Although this strain is poorly characterized in terms of its virulence factors, it has been determined that F. tularensis LVS does express a modified O antigen, antigenically distinct from wild-type O antigen. Nonetheless, F. tularensis LVS is highly virulent in mice.

The live attenuated strains of the present invention are not highly virulent. The virulence is substantially reduced to a level where the organism is not considered pathogenic by those of ordinary skill in the art. As used herein, avirulent means, for example, that the strain is non-lethal when administered to mice as described in the examples below.

The live attenuated strains of F. tularensis of the invention have a reduced expression of wild-type O antigen as compared to pathogenic F. tularensis. As used herein, wild-type O antigen refers to O antigen polysaccharide of F. tularensis LPS, the polysaccharide having identical tetrasaccharide repeats of the rare sugars 2-acetamido-2,6-dideoxy-D-glucose (D-QuiNAc), 4,6-dideoxy-4-formamido-D-glucose (D-Qui4NFm), and 2-acetamido-2-deoxy-D-galacturonamide (D-GalNAcAN). Vinogradov E V et al. (1991) Carbohydr Res 214:289-97; Conlan J W et al. (2002) Vaccine 20:3465-71. The repeating structure of wild-type O antigen can be represented as
[(1→2) (D-Qui4NFm) (1→4) (D-GalNAcAN) (1→4) (D-GalNAcAN) (1→3) (D-QuiNAc) (1→2)]
   wherein (1→2), (1→3), and (1→4) represent types of glycosidic linkages. Prior J L (2003) J Med Microbial 52:845-51. This wild-type O-antigen polysaccharide with repeating tetrasaccharide structure gives rise to a ladder pattern on SDS-PAGE, as depicted in the examples below. At least one monoclonal antibody specific for this antigen is commercially available (MAb2033, Abcam, Inc., Cambridge, Mass.).

As used herein, reduced expression of wild-type O antigen refers to any phenotype characterized by a reduced amount of wild-type O antigen expressed by F. tularensis. In one embodiment reduced expression of wild-type O antigen refers simply to expression of wild-type O antigen in a reduced amount compared to pathogenic F. tularensis. Without meaning to be bound to a particular mechanism, such embodiment can be viewed essentially as reflective of a quantitative change in expression of wild-type O antigen relative to pathogenic F. tularensis. In one embodiment reduced expression of wild-type O antigen refers to expression of an O antigen other than wild-type O antigen. Without meaning to be bound to a particular mechanism, this latter embodiment can be viewed essentially as reflective of a qualitative change in expression of O antigen relative to pathogenic F. tularensis. For example, in the latter embodiment O antigen expression still could be high, but the wild-type O antigen is no longer expressed.

The reduced expression is measured relative to expression of wild-type O antigen by pathogenic F. tularensis. In one embodiment the reduced expression is less than 50 percent relative to pathogenic F. tularensis. In one embodiment the reduced expression is less than 25 percent relative to pathogenic F. tularensis. In one embodiment the reduced expression is less than 10 percent relative to pathogenic F. tularensis. In one embodiment the reduced expression is less than 5 percent relative to pathogenic F. tularensis. In one embodiment the reduced expression is less than 1 percent relative to pathogenic F. tularensis. In one embodiment the reduced expression is no expression, i.e., 0 percent relative to pathogenic F. tularensis.

Expression of wild-type O antigen can be measured using any suitable method. In one embodiment a preparation of F. tularensis, whole cell lysate thereof, or membrane fraction thereof is contacted with an antibody that binds wild-type O antigen, under conditions that permit binding of the antibody to the wild-type O antigen. The amount of antibody bound by the F. tularensis, whole cell lysate thereof, or membrane fraction thereof is then measured using an assay sensitive to the presence of the antibody, e.g., enzyme-linked immunosorbent assay (ELISA) or immunoblotting. Expression of wild-type O antigen can alternatively or in addition be measured by chemical analysis of exopolysaccharide expressed by F. tularensis.

It is believed that wild-type O antigen expressed by pathogenic F. tularensis acts to conceal immunogenic epitopes, thereby promoting evasion by the pathogenic F. tularensis of immune defense mechanisms of a host. Reduction or elimination of wild-type O antigen thus is believed to unmask immunogenic epitopes of F. tularensis, thereby promoting elimination of the pathogenic F. tularensis by immune defense mechanisms of a host.

In one embodiment the reduced expression of wild-type O antigen results from a mutation of a gene affecting O-antigen biosynthesis. It was recently reported that an O-antigen biosynthesis locus has been identified in F. tularensis. The O-antigen biosynthesis locus occupies approximately 17 kb on the F. tularensis chromosome and contains fifteen tightly linked genes involved in O-antigen biosynthesis. Prior J L et al. (2003) J Med Microbiol 52:845-51; GenBank accession no. AY217763, the entire contents of which are incorporated by reference herein. The fifteen genes are all transcribed in the same direction and are likely organized as an operon. The genes in this biosynthesis gene cluster include at least the following: wbtA, wbtB, wbtC, wbtD, wbtE, wbtF, wzy, wbtG, wbtH, wbtI, wbtJ, wzx, wbtK, wbtL, and wbtM. The wbtA gene is the first gene in this locus. Putative assignment of specific functions in respect of O antigen subunit synthesis have been made for products encoded by at least the following genes: wbtA, wbtB, wbtC, wbtD, wbtE, wbtF, wbtG, wbtH, wbtI, wbtJ, wbtK, wbtL, and wbtM. Prior J L et al. (2003) supra.

As used herein, mutation of a gene affecting O-antigen biosynthesis refers to any mutation of a gene contained within the genome of F. tularensis, which gene can include but is not limited to a gene within the O-antigen biosynthesis locus, wherein the mutation alters O-antigen biosynthesis in a quantitative, qualitative, or both qualitative and qualitative manner, as compared to O-antigen biosynthesis in absence of such mutation. In one embodiment the mutation is in a gene outside of the O-antigen biosynthesis locus. In one embodiment the mutation is a mutation of a gene within the O-antigen biosynthesis locus.

When the mutation is a mutation of a gene within the O-antigen biosynthesis locus, the mutation can be in any one or more genes within the O-antigen biosynthesis locus. Thus in various embodiments when the mutation is a mutation of a gene within the O-antigen biosynthesis locus, the mutation is a mutation of any one or combination of wbtA, wbtB, wbtC, wbtD, wbtE, wbtF, wzy, wbtG, wbtH, wbtI, wbdJ, wzx, wbtK, wbtL, and wbtM.

As mentioned above, the wbtA gene is the first gene in the O-antigen biosynthesis locus. The wbtA gene is reported to have a nucleotide sequence provided as follows (SEQ ID NO:1; corresponding to nucleotides 975-2711 disclosed in GenBank accession no. AY217763).

atgtctttct acgataatag aacgcttaat ttcgtggtaa taatagtttt aactattatt
actgttaatt ggactttcta tattttcaag caagatgtta atttacattt tttacttgca
ttagttttgc tgagatgctt gtcatctttt ttactactta gagattatat ggctagttgg
cgtaagtcga ctcaaaaaac ttttttacgt aaggctttta ttaatttgcc agtatttttc
atagtggcat tattttttta tggcaaagtc actttttcgt tgatattctc tgagttttta
ttttatgttt ttttgatcag tttaagtgtc tacttttatt ggtatttgat gaacagagga
tcagtggata aaagtaaaac tgcggttatt tatggtgcag gtgctgcagg aacaaagatt
gctcaagaac ttgcttctgc tggttatcgc atcaaatgtt ttgttgatga caatgaaact
ttacaaaaaa gaagtattga tagtaaaaag gttctatcta aagctgaatt aacaaaacta
ttgctatcta gtagatttga ccttttggtt attgcattgc caagaaatgc aaaccaagta
gtcaaaaata tatataaaga atttgaaaag gattttaatc agattagaat tatgccgcct
cttgaggaaa ttcttcaaga tgagaatttt atgtcacagt tgaagcctgt ttcactctat
gatctattag cgcgtgatac taagagttta gataaagaat ctatctctaa ttttatcaaa
aataaggtgg tgctagtcac aggagctgga ggtagtatag gttctgaaat agtacatcaa
tgtatcaagt atcaggcaaa agagttgata ttggttgatc atagtgagtt taacttatat
aaaattactg aggagtgtag tcattttaat atcaatagtg tgctatgttc tgtttgtgat
agaaaagcat tggctgaggt ttttcaaaag tatactccaa atatagtatt tcatgctgct
gcctacaagc atgttccctt agttgaggag aatatctcta gagcaattag aaataatatc
ttaggtacta agaatgctat agatctggct atagaagctg gtgttgagtc atttatattg
atttccactg ataaagcagt gcgaccaacg aatgttatgg gggctaccaa gagagtttgt
gagctgtatt tacagaatgt tgatcccaaa aataccaagc ttgctgcagt gcgttttggt
aatgtgcttg gtagtagtgg cagtgtgatt ccaaaatttg aagagcaaat aagaaaaggt
ggtcctgtta cagttactca tcctgaaatt acacgttatt ttatgttgat accagaagct
tgtgaactgg tcctacaagc tggtgctatt gcaaaaaatt cagaggtctt tgtcttagat
atggggcaac ctgtcaagat tattgatctt gctaaacaat ttattagact ttctggtaga
ggtgatattg atattaaaat agttggtttg cgtccaggag agaaacttta cgaagagctt
ttgatagagg aagatgatgt tagtaccgac tataaagata tttttattgg tagaaggact
ttttacgata ttaatactct aaaccaagat attgaatcgt tgatcaagga tgatgttgat
cagcttgtga tattaaagaa aattgttccg gaatttgaac atagattgaa tgggtag

WbtA, the protein encoded by the wbtA gene, is reported to have an amino acid sequence provided as follows (GenBank accession no. AAS60264.1; SEQ ID NO:2).

MSFYDNRTLN FVVIIVLTII TVNWTFYIFK QDVNLHFLLA LVLLRCLSSF LLLRDYMASW
RKSTQKTFLR KAFINLPVFF IVALFFYGKV TFSLIFSEFL FYVFLISLSV YFYWYLMNRG
SVDKSKTAVI YGAGAAGTKI AQELASAGYR IKCFVDDNET LQKRSIDSKK VLSKAELTKL
LLSSRFDLLV IALPRNANQV VKNIYKEFEK DFNQIRIMPP LEEILQDENF MSQLKPVSLY
DLLARDTKSL DKESISNFIK NKVVLVTGAG GSIGSEIVHQ CIKYQAKELI LVDHSEFNLY
KITEECSHFN INSVLCSVCD RKALAEVFQK YTPNIVFHAA AYKHVPLVEE NISRAIRNNI
LGTKNAIDLA IEAGVESFIL ISTDKAVRPT NVMGATKRVC ELYLQNVDPK NTKLAAVRFG
NVLGSSGSVI PKFEEQIRKG GPVTVTHPEI TRYFMLIPEA CELVLQAGAI AKNSEVFVLD
MGQPVKIIDL AKQFIRLSGR GDIDIKIVGL RPGEKLYEEL LIEEDDVSTD YKDIFIGRRT
FYDINTLNQD IESLIKDDVD QLVILKKIVP EFEHRLNG

The WbtA protein belongs to the subfamily of dehydratases that catalyzes the conversion of UDP-glucose to UDP-4-keto 4,6 dideoxy glucose, a key step in the synthesis of the D-QuiNAc sugar of the F. tularensis O-antigen repeat unit. This family of enzymes is encoded by nearly all bacterial polysaccharide biosynthesis loci and is involved in the synthesis of complex nucleotide-linked monosaccharides from less complex sugars. The WbtA protein has similarities to several members of the epimerase/dehydratase family, with closest identities to proteins encoded by the pglF gene of Campylobacter jejuni, the wbpM gene from Pseudomonas spp., and the yveM gene from Bacillus subtilis. Belanger Met al. (1999) Microbiology 145(Pt 12):3505-21. Of the epimerase/dehydratase subfamily, the WbpM protein of P. aeruginosa is the best characterized. In Pseudomonas, this dehydratase is reported to be highly conserved and essential in all serotypes that contain D-QuiNAc or its derivative in its O-antigen repeat unit. The WbtA protein was also found to be highly conserved among the sequenced type A and type B strains of F. tularensis, consistent with a key role of this dehydratase in virulence of F. tularensis.

In one embodiment the mutation is a mutation of wbtA.

Mutations useful according to the invention can be made by insertion, deletion, or substitution of nucleotide sequence in the F. tularensis chromosome such that the mutation reduces or abolishes expression of wild-type O antigen. In one embodiment the mutation includes an insertion into the wbtA gene.

Having now identified the O antigen as a virulence factor of F. tularensis and having available the complete genomic sequence of F. tularensis, including in particular the O-antigen biosynthesis locus (GenBank accession no. AY217763), mutations can be introduced in a site-directed or shotgun manner, followed by screening and verification on the basis of reduced or absent O-antigen expression compared to control F. tularensis. Mutations useful according to the invention include, in one embodiment, any mutation directed to one or more genes involved in the biosynthesis of O antigen. In one embodiment a site-directed mutation is introduced into any one or more genes in the O-antigen biosynthesis locus. In one embodiment a site-directed mutation is introduced into wbtA. Methods for introducing site-directed mutations, i.e., site-directed mutagenesis, are well known in the art and include homologous recombination.

Deletion mutants can be constructed using any of a number of techniques well known and routinely practiced in the art. In one example, a strategy using counterselectable markers can be employed which has commonly been utilized to delete genes in many bacteria. For a review, see, for example, Reyrat J M et al. (1998) Infect Immun 66:4011-7, incorporated herein by reference. In this technique, a double selection strategy is often employed wherein a plasmid is constructed encoding both a selectable and counterselectable marker, with flanking DNA sequences derived from both sides of the desired deletion. The selectable marker is used to select for bacteria in which the plasmid has integrated into the genome in the appropriate location and manner. The counterselectable marker is used to select for the very small percentage of bacteria that have spontaneously eliminated the integrated plasmid. A fraction of these bacteria will then contain only the desired deletion with no other foreign DNA present. The key to the use of this technique is the availability of a suitable counterselectable marker.

In another technique, the cre-lox system is used for site-specific recombination of DNA. The system consists of 34-base pair lox sequences that are recognized by the bacterial cre recombinase gene. If the lox sites are present in the DNA in an appropriate orientation, DNA flanked by the lox sites will be excised by the cre recombinase, resulting in the deletion of all sequences except for one remaining copy of the lox sequence. Using standard recombination techniques, it is possible to delete the targeted gene of interest in the Francisella genome and to replace it with a selectable marker (e.g., a gene coding for kanamycin resistance) that is flanked by the lox sites. Transient expression (by electroporation of a suicide plasmid containing the cre gene under control of a promoter that functions in Francisella) of the cre recombinase should result in efficient elimination of the lox-flanked marker. This process would result in a mutant containing the desired deletion mutation and one copy of the lox sequences.

Shotgun mutation involves insertion, deletion, or substitution of nucleotide sequence at random sites within the genome. Such mutations can be made, for example, using a transposon-mediated insertion method described in the Examples below.

As described in more detail in the Examples below, a mini mariner transposon was constructed and used to randomly mutagenize the Francisella chromosome in F. tularensis LVS. These studies led to the identification of a mutant with altered surface polysaccharide expression, the insertion of which was mapped to the wbtA gene of the O-antigen cluster of Ft. LVS. The Ft. LVSΩwbtA mutant was shown to exhibit similar doubling times when compared to the wild-type LVS strain in in vitro growth experiments. Remarkably, the Ft. LVSΩwbtA strain was demonstrated to be severely attenuated in a murine tularemia model, even at the highest challenge dose administered. Furthermore, this mutant was also shown to be significantly reduced in its ability to disseminate when compared to the wild-type LVS strain. The inability of the mutant strain to cause disease and disseminate correlated with essentially complete abrogation of the surface wild-type polysaccharide expression in the Ft. LVSΩwbtA strain as revealed by immunoblot and electron microscopy studies.

Although mutation of F. tularensis LVS was found to be useful according to the invention, mutation of other strains of F. tularensis, including in particular highly virulent strains of F. tularensis, are contemplated by the invention. In one embodiment the starting strain is type A F. tularensis. In one embodiment the starting strain is type B F. tularensis.

In one aspect the invention is a vaccine composition including an effective amount of a live attenuated avirulent strain of Francisella tularensis, wherein the live attenuated avirulent strain has reduced expression of wild-type O antigen as compared to pathogenic F. tularensis, and a pharmaceutically acceptable carrier. In one embodiment the vaccine composition includes two or more individual live attenuated avirulent strains of Francisella tularensis bacteria, wherein at least one live attenuated avirulent strain has reduced expression of wild-type O antigen as compared to pathogenic F. tularensis.

As used herein, an effective amount refers to an amount effective to induce an immune response to a live attenuated strain of F. tularensis of the invention in a mammal following exposure to or administration of F. tularensis to the mammal. In one embodiment the effective amount is an amount that is effective to induce protective immunity against typical exposure to a pathogenic strain of F. tularensis. As used herein, typical exposure to a pathogenic strain of F. tularensis refers to exposure by aerosol exposure (e.g., inhalation) or by intradermal or transcutaneous introduction of a clinically relevant inoculum of pathogenic F. tularensis. As used herein, protective immunity refers to an immune response effective to prevent or ameliorate signs or symptoms of disease caused by F. tularensis, including dissemination of F. tularensis in tissues of the reticuloendothelial system. Either humoral immunity or cell-mediated immunity or both may be induced. The immunogenic response of a mammal to a vaccine composition may be evaluated, e.g., indirectly through measurement of antibody titers, lymphocyte proliferation assays, or directly through monitoring signs and symptoms after challenge with wild-type strain. The protective immunity conferred by a vaccine can be evaluated by measuring, e.g., reduction in clinical signs such as mortality, morbidity, lymphadenopathy, fever, skin ulceration, respiration, physical condition and overall health and performance of the subject.

In one embodiment the effective amount is at least 10³ colony forming units (cfu). In one embodiment the effective amount is at least 10⁴ cfu. In one embodiment the effective amount is at least 10⁵ cfu. In one embodiment the effective amount is at least 10⁶ cfu. In one embodiment the effective amount is at least 10⁷ cfu. In one embodiment the effective amount is at least 10⁸ cfu. In one embodiment the effective amount is at least 10⁹ cfu. In one embodiment the effective amount is at least 10¹⁰ cfu.

As used herein, a pharmaceutically acceptable carrier refers to any suitable nontoxic liquid, semisolid, or solid diluent or encapsulating substance that is compatible with the active ingredient and suitable for administration to a human or other mammal. The particular choice of carrier can vary depending on the intended route of administration or formulation. In one embodiment the carrier is sterile. The term carrier denotes an organic or inorganic ingredient, natural or synthetic, with which the active ingredient is combined to facilitate the application. The components of the pharmaceutical compositions of the invention also are capable of being commingled with the compounds of the present invention, and with each other, in a manner such that there is no interaction which would substantially impair the desired pharmaceutical efficacy. Pharmaceutically acceptable liquid carriers can include, without limitation, physiologic saline, water, and phosphate buffered saline.

In one aspect the invention is a method of vaccinating a mammal against a pathogenic Francisella tularensis bacteria, comprising administering to the mammal an effective amount of a live attenuated strain of Francisella tularensis bacteria, wherein the live attenuated strain has reduced expression of wild-type O-antigen as compared to pathogenic F. tularensis.

As used herein, administering refers to any suitable method of dispensing and delivering a treatment agent to the body of a subject. Suitable routes of administration include but are not limited to intradermal, intranasal, inhalation, intramuscular, intravenous, subcutaneous, mucosal, and enteral. Inhalation specifically includes aerosol administration to lung. Intranasal administration includes liquid (e.g., nose drops) and aerosol to nasal mucosa. Enteral specifically includes but is not limited to oral.

As used herein a mammal refers to a human or a non-human mammal.

The invention in one aspect is a method for identifying an agent that binds to and inhibits activity of an enzyme involved in biosynthesis of wild-type O antigen of a pathogenic strain of Francisella tularensis. The method includes the steps of contacting an enzyme involved in biosynthesis of wild-type O antigen of a pathogenic strain of Francisella tularensis with a test agent; measuring activity of the enzyme in presence of the test agent; and determining whether the test agent binds to and inhibits activity of the enzyme. As used herein, an enzyme involved in biosynthesis of wild-type O antigen of a pathogenic strain of Francisella tularensis is an enzyme encoded by a gene in the O-antigen biosynthesis locus of a pathogenic strain of F. tularensis. In various embodiments the enzyme is any one of WbtA, WbtB, WbtC, WbtD, WbtE, WbtF, Wzy, WbtG, WbtH, WbtI, Wzx, WbtJ, WbtK, WbtL, and WbtM. In one embodiment the enzyme is WbtA. In one embodiment the contacting occurs within a live cell. In one embodiment the contacting occurs with an isolated enzyme. An isolated enzyme is an enzyme that is removed from its natural environment, e.g., removed from a cell. In one embodiment an isolated enzyme is removed from other enzymes.

As used herein, a test agent is any natural or synthetic compound. Test agents can include, without limitation, small organic molecules having molecular weight of up to 1.5 kDa, carbohydrates, saccharides (including monsoaccharides, oligosaccharides, and polysaccharides), nucleotides (including single nucleotides and analogs thereof, oligonucleotides, and polynucleotides), amino acids, peptides (including oligopeptides and polypeptides), proteins, antibodies and fragments thereof, and lipids.

Measuring activity of the enzyme can be performed directly or indirectly. Direct measurement can be performed with an isolated enzyme in the presence of suitable substrate for the enzyme under conditions otherwise suitable for activity by the enzyme. Such direct measurement entails measuring rate or extent of conversion of substrate to product by the enzyme. Indirect measurement can involve measurement of a product that is downstream of a product specifically generated by the enzyme. For example, the various enzymes encoded by genes in the O-antigen biosynthesis locus have various products, all of which contribute to generation of O antigen. O antigen thus is a downstream product of each and every such enzyme, and an indirect measurement may be made by measuring O antigen per se.

The activity of the enzyme is determined to be inhibited if its activity is reduced in the presence of the test agent as compared to its activity under similar conditions in absence of the test agent. In one embodiment the activity is reduced by at least 5 percent. In one embodiment the activity is reduced by at least 10 percent. In one embodiment the activity is reduced by at least 25 percent. In one embodiment the activity is reduced by at least 50 percent. Comparison can be made based on contemporaneous or historical control measurement made under similar conditions in absence of the test agent.

The method also involves determining whether the test agent binds to the enzyme. This can be accomplished using any suitable method and optionally can be performed apart from determining whether the test agent inhibits activity of the enzyme. In one embodiment the enzyme can be immobilized onto a surface or support and then contacted with a test agent that has been labeled, e.g., radiolabeled, such that its presence can be detected using a suitable detector. After contacting the immobilized enzyme with a source of labeled test agent, excess unbound labeled test agent is removed, for example by one or more wash steps, and remaining labeled test agent is measured. Suitable additional steps can be taken to account for nonspecific binding, for example by running a control with no enzyme present. The nonspecific binding can then be subtracted from the binding with the enzyme.

The invention in one aspect is a method for identifying an agent that binds to and inhibits activity of a gene involved in biosynthesis of wild-type O antigen of a pathogenic strain of Francisella tularensis. The method includes the steps of contacting a gene involved in biosynthesis of wild-type O antigen of a pathogenic strain of Francisella tularensis with a test agent; measuring activity of the gene in presence of the test agent; and determining whether the test agent binds to and inhibits activity of the gene. As used herein, a gene involved in biosynthesis of wild-type O antigen of a pathogenic strain of Francisella tularensis is a gene in the O-antigen biosynthesis locus of a pathogenic strain of F. tularensis. In various embodiments the enzyme is any one of wbtA, wbtB, wbtC, wbtD, wbtE, wbtF, wzy, wbtG, wbtH, wbtI, wzx, wbtJ, wbtK, wbtL, and wbtM. In one embodiment the gene is wbtA. In one embodiment the contacting occurs within a live cell. In one embodiment the contacting occurs with an isolated gene. An isolated gene is a gene that is removed from its natural environment, e.g., removed from a cell. In one embodiment an isolated gene is removed from other genes.

In one embodiment the test agent according to this aspect of the invention is a DNA or RNA molecule 10 to 2000 nucleotides long having a nucleotide sequence that is complementary to at least a portion of the gene.

Measuring activity of the gene can be performed directly or indirectly. Direct measurement can be performed, for example, by measuring the amount of RNA transcript expressed in a cell. Indirect measurement can involve measurement of a protein or polysaccharide product that is downstream of an RNA transcript specifically generated by the gene. For example, the various genes in the O-antigen biosynthesis locus have sequence-specific transcripts encoding individual enzymes, all of which contribute to generation of O antigen. O antigen thus is a downstream product of each and every such gene, and an indirect measurement may be made by measuring O antigen per se. An enzyme encoded by the gene may be detected using suitable antibodies or other agents that selectively bind to the enzyme.

The activity of the gene is determined to be inhibited if its activity is reduced in the presence of the test agent as compared to its activity under similar conditions in absence of the test agent. In one embodiment the activity is reduced by at least 5 percent. In one embodiment the activity is reduced by at least 10 percent. In one embodiment the activity is reduced by at least 25 percent. In one embodiment the activity is reduced by at least 50 percent. Comparison can be made based on contemporaneous or historical control measurement made under similar conditions in absence of the test agent.

The method also involves determining whether the test agent binds to the gene. This can be accomplished using any suitable method and optionally can be performed apart from determining whether the test agent inhibits activity of the gene. In one embodiment the gene can be immobilized onto a surface or support and then contacted with a test agent that has been labeled, e.g., radiolabeled, such that its presence can be detected using a suitable detector. After contacting the immobilized gene with a source of labeled test agent, excess unbound labeled test agent is removed, for example by one or more wash steps, and remaining labeled test agent is measured. Suitable additional steps can be taken to account for nonspecific binding, for example by running a control with no gene present. The nonspecific binding can then be subtracted from the binding with the gene.

Formulations for clinical use include the live attenuated avirulent strain of F. tularensis, alone or in combination with another agent. The other agent in one embodiment is an agent that enhances the immune response to the live attenuated avirulent strain of F. tularensis. In one embodiment the other agent is an adjuvant. As used herein, an adjuvant is an agent that stimulates the innate immune system, i.e., stimulates the immune system in a non-specific manner. Adjuvants enhance T-cell activation by promoting the accumulation and activation of other leukocytes at a site of antigen exposure. Adjuvants enhance accessory cell expression of T-cell-activating costimulators and cytokines.

In one embodiment the adjuvant is a cytokine. In one embodiment the adjuvant is interleukin 12 (IL-12). In one embodiment the adjuvant is cholera toxin subunit. In one embodiment the adjuvant is QS21. In one embodiment the adjuvant is an immunostimulatory CpG oligonucleotide.

Formulations for clinical use can also include combination with another vaccine antigen.

The formulations of the invention are administered in pharmaceutically acceptable solutions, which may routinely contain pharmaceutically acceptable concentrations of salt, buffering agents, preservatives, compatible carriers, and optionally adjuvants and other therapeutic ingredients.

For use in therapy, an effective amount of the live attenuated avirulent strain of F. tularensis can be administered to a subject by any mode that delivers the live attenuated avirulent strain of F. tularensis to the desired surface. Administering the pharmaceutical composition of the present invention may be accomplished by any means known to the skilled artisan. Routes of administration include but are not limited to oral, parenteral, intramuscular, intranasal, intradermal, sublingual, intratracheal, inhalation, ocular, vaginal, and rectal. Preferred routes are by injection or by inhalation. Vaccine compositions are well known in the pharmaceutical arts.

For oral administration, the compounds (i.e., live attenuated avirulent strain of F. tularensis and other therapeutic agents) can be formulated readily by combining the live attenuated avirulent strain of F. tularensis with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Nasal delivery of a pharmaceutical composition of the present invention is also contemplated. Nasal delivery allows the passage of a pharmaceutical composition of the present invention to the nasal mucosa, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the vaccine in water-soluble form. Additionally, suspensions may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### Examples

The following Examples describe the crucial role of surface polysaccharide expression in the pathobiology of Francisella tularensis LVS. A mariner transposon mutagenesis strategy using Himar1 was exploited to generate an insertion mutant in the polysaccharide coding region of F. tularensis LVS. Southern blot and genomic sequencing mapped one of the insertions to be in the wbtA gene of the O-antigen locus of this intracellular pathogen. The wbtA gene purportedly encodes a dehydratase, and inactivation of this gene resulted in the complete disruption of the surface polysaccharide expression as determined by western blot, ELISA and immune electron microscopy. The Ft. LVSΩwbtA strain was demonstrated to be severely debilitated in its ability to disseminate and remained avirulent when tested in a murine model of tularemia via the intradermal and intranasal route. These studies demonstrate a pivotal role of the dehydratase gene in the surface polysaccharide expression and pathogenesis of this microbe. This is the first study in F. tularensis to demonstrate that inactivation of an enzyme responsible for synthesis of a structural product results in attenuation of this highly infectious bacterium. An attenuated mutant strain of the current live vaccine strain of F. tularensis that is devoid of its O-side chain/capsule (O-antigen) is described.

### Example 1

### Himar1 Transposon Mutagenesis of F. tularensis LVS and Identification of Polysaccharide Mutants

### Materials and Methods

Bacteria and growth conditions. F. tularensis LVS (kindly provided by Dr. Karen Elkins, U.S. Food and Drug Administration, Bethesda, Md.) was cultured on cysteine heart agar supplemented with 1% hemoglobin (CHAR) for 72 hours (hrs) at 37° C. in 5% CO₂. Colonies were grown in modified Mueller-Hinton broth (Difco, Detroit, Mich.) supplemented with ferric PPi and IsoVitaleX® (Becton Dickinson, Cockeysville, Md.). The doubling time of insertion mutants Ft. LVSΩwbtA and Ft. LVSΩcapB was compared to the parent strain in vitro using modified Mueller-Hinton broth following inoculation of the cultures at an approximate OD₆₂₀ 0.05. All growth experiments were performed in triplicate.

Construction of Francisella transposon. Mariner transposon constructs for the mutagenesis of F. tularensis LVS were constructed as follows. 1) To account for mariner insertions in both the 5′ and 3′ orientation in the Francisella genome, either a 241 or 125 by native groEL promoter sequence was ligated to the Tn903-derived kanamycin ORF by SOEing PCR reaction. The primer pairs used were SD52 (TCTAGCGGCCGCACTATACCCTTCAAGCTTTG (SEQ ID NO:3))+SD38 (CGTTGAATATGGCTCATAACAATCTTACTCCTTTG (SEQ ID NO:4)) for the 241 by promoter and SD48 (TCTAGCGGCCGCTTGAAAATTTTTTTTTTGAC (SEQ ID NO:5))+SD38 for the 125 by promoter. Both of these promoter elements were then individually fused to the Tn903-derived kanamycin ORF which was PCR amplified using the primer pairs SD39 (GGAGTAAGATTGTTATGAGCCATATTCAACGG (SEQ ID NO:6)) and SD40 (GTACGGATTCCAACCCTGAAGCTTGCTTGC (SEQ ID NO:7)). The Himar1 inverted terminal repeats (ITR) were added to the ends of each gene fusion by cloning the fragments from the previous step into a vector that has the ITRs with cloning sites in between to generate the mini mariner transposons, SD1 (ITR-241Kan-ITR) and SD2 (ITR-125kan-ITR), respectively. The primer SD65 (GATTCCGGATAACAGGTTGGC (SEQ ID NO:8)) was used to PCR out the transposons and add a BspEI site to their ends. In the final step, the mini mariner transposons, SD1 and SD2, were cloned into the XmaI site of the Himar1 C9 transposase-expressing plasmid pSC186.

Transposon mutagenesis of F. tularensis LVS. For mutagenesis of F. tularensis LVS, the suicide delivery plasmids were introduced into the bacteria by electroporation as described previously (Baron et al. (1995) Methods Mol Biol 47:149-54). Following electroporation, the cells were resuspended in 1 ml of tryptic soy broth supplemented with cysteine (TSB-C), incubated for 4 hr at 37° C. on a rotary shaker, and then plated on cysteine heart agar media supplemented with 2% bovine hemoglobin and 5 μg/ml kanamycin. Individual kanamycin-resistant colonies appeared after 2-3 days of incubation at 37° C. Chromosomal DNA was prepared from each individual mutant and the insertion site of each transposon mutant was identified by genomic sequencing as previously described (Kawula et al. (2004) Appl Environ Microbiol 70:6901-4).

Southern blot hybridization. Chromosomal DNA isolated from the wild-type and Himar1 insertion mutants was digested with NcoI and probed with a digoxigenin-labeled Himar1 DNA probe. 5 μg of the chromosomal DNA was digested with NcoI overnight, separated on a 1% agarose gel, transferred onto a Zeta-Probe (BioRad, Hercules, Calif.) membrane and probed using digoxigenin-labeled Tn903 kanamycin resistance gene.

Quantitative Real-Time PCR. Total RNA was prepared from Ft. LVS by lysing pelleted bacteria in TRIzol® reagent (Invitrogen, Carlsbad, Calif.). Residual genomic DNA was removed by DNaseI treatment with subsequent further purification of the RNA by RNeasy® (Qiagen, Valencia Calif.). cDNA was generated from RNA using the Omniscript® kit (Qiagen). Control reactions proved the lack of any contaminating genomic DNA in the purified RNA. qRT-PCR was accomplished using a DNA Engine Opticon 2® (MJ Research, Waltham, Mass.).

### Results

Himar1 transposon was utilized for the random mutagenization of F. tularensis LVS chromosome to isolate surface polysaccharide mutants. One of the major advantages of Himar1 transposon is the minimal requirement of only the dinucleotide TA in the target sequence for transposition. To generate random insertion mutants in F. tularensis LVS, two suicide delivery plasmids, pSD1 and pSD2, were constructed with the following characteristics: 1) A Tn903-derived kanamycin ORF under the control of the F. tularensis LVS groEL promoter; 2) Himar1 inverted terminal repeats (ITR) flanking the kanamycin resistance gene to generate the mini mariner transposon; and 3) a C9 transposase gene which allowed the efficient transposition of the Himar1 mini transposon into the chromosome.

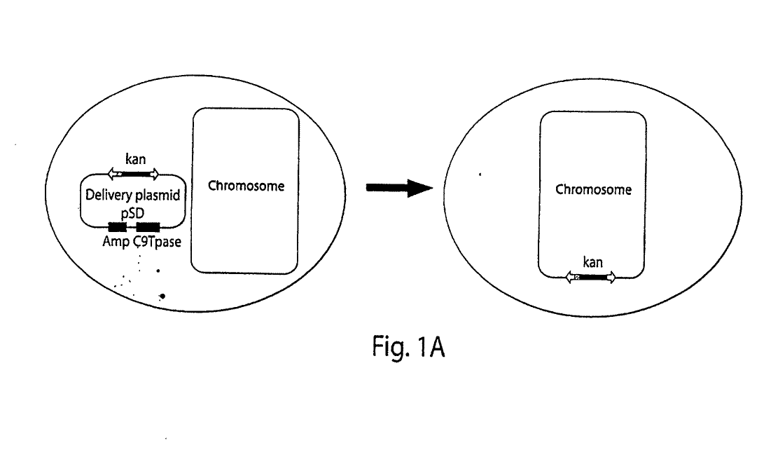

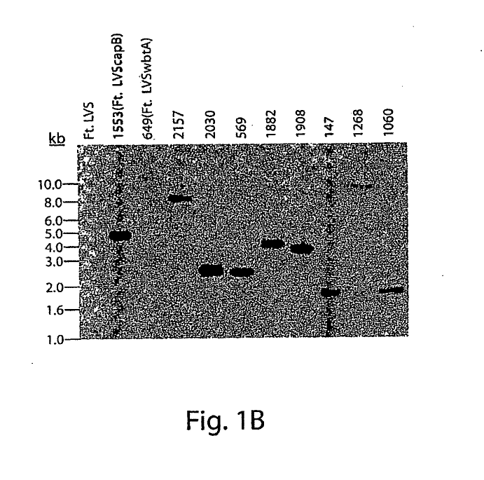

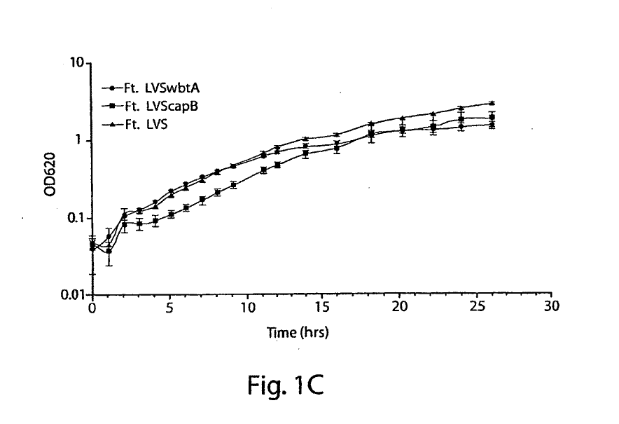

To create insertion mutants, the suicide delivery plasmids pSD1 and pSD2 were electroporated into electrocompetent F. tularensis LVS as described previously and the transformants plated on CHAH plates supplemented with kanamycin (Baron et al. (1995) Methods Mol Biol 47:149-54). The delivery plasmid did not replicate in the host and resistance to kanamycin was only achieved if a transposition event has occurred (FIG. 1A). Ten kanamycin-resistant mutants from each of the two delivery plasmid transformant pools were analyzed to determine the orientation and randomness of transposon insertion into the Francisella chromosome.

Analysis of pSD1-derived transformants identified the transposition to be random and in both orientations. Genomic sequencing identified one of the insertions to be in the putative O-antigen biosynthesis cluster. The insertion was mapped to be in 5′ end of the wbtA gene at nucleotide (nt) position 14 (amino acid position 4 from the N-terminal end of the WbtA ORF (GenBank Accession No. AY217763, the content of which is incorporated by reference). The wbtA gene represents the first of the fifteen gene O-antigen biosynthesis cluster of F. tularensis and has been proposed to encode for a dehydratase (Prior et al. (2003) J Med Microbiol 52:845-51). The wbtA gene product catalyzes the conversion of UDP-glucose to UDP-4-keto 4,6-dideoxyglucose, a quintessential step in the formation of QuiNAc (2-acetamido-2,6-dideoxy-D-glucose) which is one of the complex sugars that comprise the tetrasaccharide repeating unit of the O-antigen in F. tularensis (Vinogradov et al. (1991) Carbohydr Res 214:289-97, Conlan et al. (2002) Vaccine 20:3465-71, Prior et al. (2003) J Med Microbiol 52:845-51).

Unlike the pSD1-derived insertion mutants, analysis of the pSD2-derived kanamycin-resistant mutants revealed all the insertions to be in the plus orientation. One likely explanation for this result is that the 125 by GroEL promoter element is not sufficient to drive the expression of the downstream kanamycin ORF. Thus, the expression of kanamycin ORF was dependent on the promoter of the resident gene into which the Himar1 transposon had been inserted. These results indicated that the suicide delivery plasmid pSD2 would not be a vector of choice to generate a saturated insertion mutant library of F. tularensis LVS. Nonetheless, one of the pSD2-derived insertions was identified to be in a Bacillus anthracis capB gene homolog. The CapB protein belongs to a family of ADP-forming amide bond ligases and is key in the formation of γ-polyglutamic acid-like capsule. Genomic sequencing mapped the insertion to be in the C-terminus of the F. tularensis LVS capB homolog at nt position 1038 (GenBank Accession No. AY217763). Based on the implication of the role of WbtA and the CapB proteins in polysaccharide biosynthesis, these two mutants were used in subsequent analyses.

Genetic characterization of Himar1 insertion mutants was performed. Southern blot analysis indicated a single transposition event of the Himar1 transposon in all the pSD1 and pSD2 derived insertion mutants examined, including that of the wbtA (Ft. LVSΩwbtA) and capB (Ft. LVSΩcapB) (FIG. 1B). The stability of the Himar1 Tn insertions in the chromosome was examined by isolating chromosomal DNA and sequencing the transposon-chromosome junctions from mutant strains passaged daily for two weeks in media without kanamycin. The initial insertion sites were found to be maintained in all the mutants analyzed. Determination of the transcriptional status of the genes downstream of the transposon by quantitative real time-PCR (qRT-PCR) analysis indicated no decrease in the level of these transcripts. Furthermore, in vitro growth studies with Ft. LVSΩwbtA and Ft. LVSΩcapB mutants indicated no obvious growth defect of the mutants when compared to parent strain under these conditions (FIG. 1C).

### Example 2

### Immune Electron Microscopy

### Materials and Methods

Immune Electron Microscopy (IBM). Bacterial cultures were harvested from CHAH plates following a 72 hr growth at 37° C. in 5% CO₂. The cultures were subjected to three washes to remove residual media component and resuspended in 1× phosphate buffered saline (PBS). 10 μl a of the suspension were spotted onto formvar-carbon coated copper grids and incubated for 30 minutes, followed by blocking for 20 min. in 0.5% fish gelatin (FSG) at room temperature (RT). The copper grids were then floated on 20 μl spots of primary antibody diluted in 0.5% FSG at a concentration of 1/20 for 30 min. at RT. Following incubation with primary antibody, the copper grids were washed by floating the grids on 1× PBS for 10 min. The grids were then incubated with the secondary antibody tagged with 15 nanomoles protein A gold (PAG) for 10 min. When using the mouse MAb 2033, an additional step using bridging antibody (rabbit anti-mouse) was performed prior to incubation with the secondary antibody. After incubation with the secondary antibody, the copper grids were first washed three times with 1× PBS followed by six washes for 10 min with sterile water prior to incubation with 1% uranyl acetate for 30 seconds. The grids were then subjected to final wash with sterile water, dried, and examined using a JOEL 1200EX transmission electron microscope. Images were recorded at a primary magnification of 30,000.

### Results

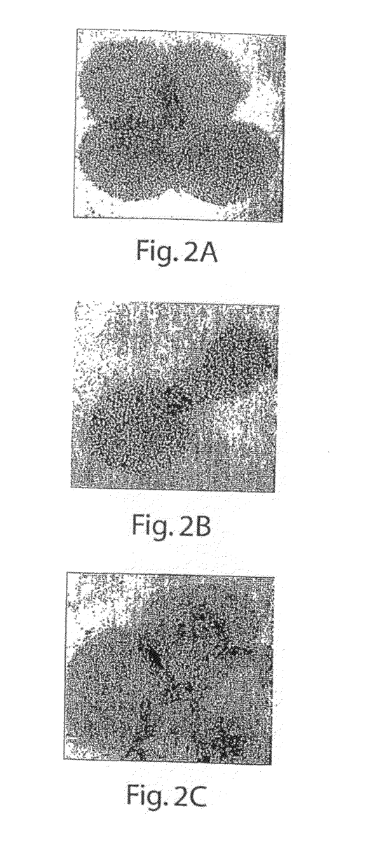

Negative contrast IEM (nc-IEM) showed extensive immunogold label on the surface of both the Ft. LVS and Ft. LVSΩcapB strains when incubated with monoclonal antibody MAb2033 (FIG. 2A and FIG. 2B). The epitope of MAb2033 is said to be on the O-antigen polysaccharide. In striking contrast, the Ft. LVSΩwbtA mutant showed a complete loss of this immunogold label (FIG. 2C). Similar results were obtained when MAb2034, the binding site of which is proposed to be on the LPS, was used.

### Example 3

### Biochemical Characterization of Himar1 Insertion Mutants

### Materials and Methods

Outer membrane preparation. The bacterial pellet was suspended in a lysis buffer (0.05 M sodium phosphate, 0.15 M NaCl, and 0.01 M EDTA adjusted to pH 7.4), and incubated at 60° C. for 30 min. The suspension was subjected to mild shearing by passage through a 25-guage hypodermic needle by manual pressure and organisms were pelleted from the suspension by centrifugation at 12,000×g at 4° C. for 20 min followed by centrifugation of this supernatant fluid at 80,000×g at 4° C. for 2 hr.

Immunoblot Analysis. Outer membranes or bacterial cell lysates (10 μl) from each strain were suspended in 1× Laemmli sample buffer, heated at 100° C. for 10 min. and subjected to SDS-PAGE on a 4% -20% gradient gel. For immunoblot analysis, the bands were transferred overnight onto an Immobilon™-P transfer membrane (Millipore, Billerica, Mass.) using Towbin buffer (25 mM Tris-HCl, 192 mM glycine, 20% methanol). Following transfer, the transfer membranes were blocked in 1% casein and probed with respective primary antibodies at a dilution of 1:3,000 in 1× casein at room temperature for 1 hr. Monoclonal antibodies MAb 2033 and MAb 2034 were purchased from Abcam, Inc. (Cambridge, Mass.). The rabbit polyclonal F. tularensis LVS antiserum used in this study was generated using the Classic-Line Protocol of Lampire Biological Laboratories. The appropriate secondary antibodies conjugated to alkaline phosphatase were used at a dilution of 1:2,000 in 1× casein for 1 hr at room temperature. The blots were developed using the alkaline phosphatase detection kit from Novagen (Madison, Wis.) according to the manufacturer's protocol. After sufficient development the transfer membrane was washed with distilled water, dried with adsorbent paper, and photographed.

Immunoblot Adsorption Analysis. To perform adsorption studies, two flasks, each containing 50 ml cultures of the parent and the two mutant (Ft. LVSΩcapB and Ft. LVSΩwbtA) strains were grown overnight on a shaker at 37° C. Following overnight growth, the set of 50 ml cultures for each of the strains was washed 3 times with 1× PBS to remove residual media components and pelleted at 4000 rpm using a table-top centrifuge.

To deplete the anti-FT serum of the surface antibodies expressed by the parent and mutant strains, one of the two 50 ml pellets from each culture set was resuspended in 10 ml of sterile 1× PBS supplemented with 100 μl of the anti-FT serum. The suspensions were incubated on a rotary shaker at 37° C. for 1 hr, following which the cells were spun down and the supernatant removed. This supernatant was then utilized to resuspend the second pellet of each of the respective cultures and adsorptions repeated. Following this second round of adsorption, the cultures were spun down and the supernatant obtained was used as adsorbed serum in immunoblot analysis.

### Results

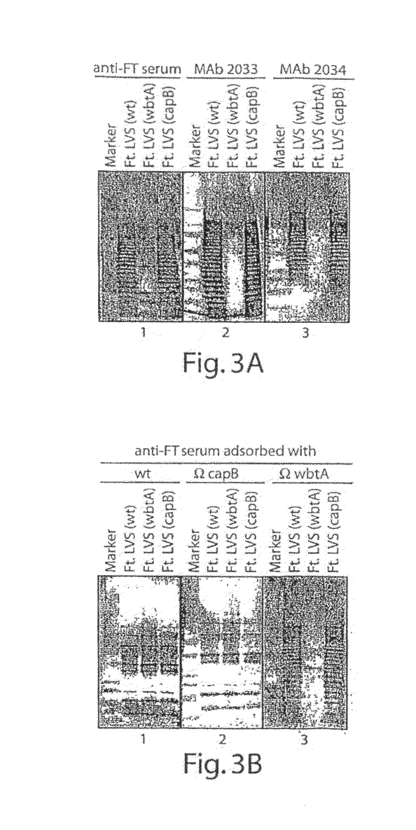

The expression status of the surface polysaccharides in the wild-type and the mariner mutant strains were further scrutinized by a series of immunoblot analyses. For immunoblot analysis, the whole cell lysates from the Ft. LVS and the mutants were prepared and analyzed as described above. Immunoblots performed with the whole organism serum (anti-FT serum) and the two MAbs (2033 and 2034) revealed a characteristic laddering O-antigen pattern with whole cell lysates of the Ft. LVS and Ft. LVSΩcapB strain (FIG. 3A, panels 1, 2 and 3). In striking contrast, a complete loss of the ladder pattern was observed when whole cell lysate from Ft. LVSΩwbtA strain was utilized (FIG. 3A, panels 1, 2 and 3). Distinct bands in Ft. LVSΩwbtA mutant lane were identified when immunoblots were probed with anti-FT serum (FIG. 3A, panel 1). These bands most likely represent protein antigens of F. tularensis LVS.

The lack of O-antigen expression was further demonstrated by generating specific antiserum to the mutant by adsorbing the anti-FT serum with the Ft. LVSΩwbtA mutant strain, so that all of the antibodies to surface molecules expressed by the Ft. LVSΩwbtA strain were removed. Therefore, following these adsorptions, only antibodies to molecules expressed by the wild-type strain but not the mutant strain remained. Similar adsorption studies were performed with the Ft. LVS and Ft. LVSΩcapB strain as controls for the initial adsorption studies. Immunoblot analyses indicated a complete loss of the O-antigen polysaccharide ladder when probed with either the Ft. LVS or the Ft. LVSΩcapB adsorbed anti-FT serum (FIG. 3B, panels 1 and 2). As identified previously, protein antigen bands of similar molecular size were observed (FIG. 3B, panels 1 and 2). The laddering polysaccharide pattern was regained in the whole cell lysates of the wild type and Ft. LVSΩcapB strain when Ft. LVSΩwbtA-adsorbed anti-FT serum was used in similar immunoblot analysis (FIG. 3B, panel 3).

No O-antigen polysaccharide-specific reaction was observed when the cell lysates of Ft. LVSΩwbtA strain were analyzed with the any of the adsorbed serums tested (FIG. 3B).

To ascertain whether the abrogation of O-antigen polysaccharide expression in the Ft. LVSΩwbtA strain was due solely to inactivation of the wbtA gene, the phenotype was complemented by introducing an intact copy of the homologous wbtA gene in trans. Immunoblot analyses showed that the complemented strain was rescued for its ability to express O-antigen polysaccharide, as evidenced by reconstitution of the polysaccharide ladder. Surface expression of O-antigen polysaccharide in the complemented strain was confirmed by immunogold labeling and ELISA inhibition studies.

### Example 4

### Virulence of Ft. LVSΩwbtA is Attenuated Compared to Ft. LVS

### Materials and Methods

Mice Challenge studies. Six- to eight-week-old male BALB/cByJ mice (stock no. 001026) were purchased from the Jackson Laboratory (Bar Harbor, Me.). The mice were caged in microisolators and housed in the animal facility of Harvard Medical School in a pathogen-free environment. Animals were anesthetized by xylazine/ketamine (Webster Veterinary Supply, Inc., Sterling, Mass.) in PBS prior to challenge experiments. The intradermal (i.d.) LD₅₀ and the intranasal (i.n.) LD₅₀ of F. tularensis LVS with male BALB/cByJ were determined by Reed and Muench method to be 1.0×10⁶ cfu and 858 cfu, respectively. In a separate study the virulence potential of the Francisella tularensis LVS wbtA mutant was determined by challenging BALB/cByJ mice cohort (n=8 unless otherwise stated) by various routes over a range of doses. One group of mice were challenged by i.d. injection with 3.7×10³ cfu to 3.7×10⁸ cfu of wbtA mutant in 100 μl physiological saline, inoculated into the skinfold of the shaved mid-belly. A second group of mice were challenged intranasally with 3.7×10¹ cfu to 3.7×10⁷ cfu of wbtA mutant, instilled intranasally in 50 μl total volume (25 μl per nostril). A third group of mice were challenged intraperitoneally with 4.0×10¹ cfu to 4.0×10⁷ cfu of wbtA mutant in 100 μl. The actual inoculum concentration was determined by plating 10-fold serial dilution on CHAH plates. Colonies were counted after 48-72 hr of incubation at 37° C. All the challenged mice were monitored for a 28 day period. Mice were monitored for signs of discomfort and stress, and animals that appeared moribund during these experiments were humanely euthanized by asphyxiation with CO₂.

### Results

The virulence potential of Ft. LVSΩwbtA strain was determined in intradermal, intranasal, and intraperitoneal models of tularemia using male BALB/c mice. Mice (n=8 per group) were challenged with 10-fold increasing doses of the Ft. LVSΩwbtA strain ranging from 3.7×10³ to 3.7×10⁸ cfu for intradermal challenge, 3.7×10¹ to 3.7×10⁷ cfu for intranasal challenge, and 4.0×10⁰ to 4.0×10⁷ cfu for intraperitoneal challenge. As shown in Table 1, all mice challenged intradermally with Ft. LVSΩwbtA survived, even at the highest dose (10⁸ cfu), whereas the LD₅₀ of the parent LVS strain was ˜10⁶ cfu. Intranasal challenge with the Ft. LVSΩwbtA strain produced similar results, with the exception of one death in the highest dose challenge group of 10⁷ cfu, and defined the LD₅₀ of the parent strain as ˜10³ cfu (Table 2). The mean time to death (MTD) of this group was determined to be >19 days, which was significantly delayed when compared to the MTD of 5 days when naive mice were challenged at a similar dose with the F. tularensis LVS parent strain in a separate study.

The attenuation status of the mutant was further scrutinized with the even more sensitive intraperitoneal challenge model of tularemia. BALB/c mice are most susceptible to this mode of challenge, with a lethal infectious dose of ˜1 cfu in intraperitoneal challenge studies. Elkins K L et al. (1992) Infect Immun 60:4571-7. As shown in Table 3, all mice challenged with the parent strain succumbed to infection even at the lowest challenge dose (4 cfu). In contrast, among mice challenged intraperitoneally with strain Ft. LVSΩwbtA, no deaths were recorded up to a dose of 10⁷ cfu.

TABLE 1
Survival of male BALB/cByJ mice following intradermal challenge
with F. tularensis LVS and Ft. LVSΩwbtA strain.

| Intradermal | | | F. tularensis | |
|---|---|---|---|---|
| Challenge Dose | F. tularensis LVS^{a} | | LVSΩwbtA^{b} | |
| (cfu) | Survival Ratio | MTD | Survival Ratio | MTD |
| 10³ | 8/8 | >21 | 8/8 | >21 |
| 10⁴ | 8/8 | >21 | 8/8 | >21 |
| 10⁵ | 8/8 | >21 | 8/8 | >21 |
| 10⁶ | 3/8 | 6.6 | 8/8 | >21 |
| 10⁷ | 0/8 | 4.6 | 8/8 | >21 |
| 10⁸ | 0/8 | 3.3 | 8/8 | >21 |

MTD, mean time to death in days
^{a}Ft. LVS challenge dose was 3.2 × cfu
^{b}Ft. LVSΩwbtA challenge dose was 3.7 × cfu

TABLE 2
Survival of male BALB/cByJ mice following intranasal challenge with
F. tularensis LVS wild type and Ft. LVSΩwbtA strain.

| Intranasal | | | F. tularensis | |
|---|---|---|---|---|
| Challenge Dose | F. tularensis LVS^{a} | | LVSΩwbtA^{b} | |
| (cfu) | Survival Ratio | MTD | Survival Ratio | MTD |
| 10¹ | 8/8 | ND | 8/8 | >21 |
| 10² | 6/8 | 15 | 8/8 | >21 |
| 10³ | 0/8 | 11.2 | 8/8 | >21 |
| 10⁴ | 0/8 | 7 | 8/8 | >21 |
| 10⁵ | 0/8 | 6 | 8/8 | >21 |
| 10⁶ | 0/8 | 5.6 | 8/8 | >21 |
| 10⁷ | 0/8 | 5.25 | 7/8 | >19 |

MTD, mean time to death in days
ND, not determined
^{a}Ft. LVS challenge dose was 3.2 × cfu
^{b}Ft. LVSΩwbtA challenge dose was 3.7 × cfu

TABLE 3
Survival of male BALB/cByJ mice following intraperitoneal challenge
with F. tularensis LVS wild type and Ft. LVSΩwbtA strain.

| Intraperitoneal | | | F. tularensis | |
|---|---|---|---|---|
| Challenge Dose | F. tularensis LVS^{a} | | LVSΩwbtA^{b} | |
| (cfu) | Survival Ratio | MTD | Survival Ratio | MTD |
| 10⁰ | 0/8 | 6 | ND | >21 |
| 10¹ | 0/8 | 5 | ND | >21 |
| 10² | 0/8 | 4.6 | ND | >21 |
| 10³ | 0/8 | 4 | 8/8 | >21 |
| 10⁴ | 0/8 | ND | 8/8 | >21 |
| 10⁵ | 0/8 | ND | 8/8 | >21 |
| 10⁶ | 0/8 | ND | 8/8 | >21 |
| 10⁷ | 0/8 | ND | 8/8 | >19 |

MTD, mean time to death in days
ND, not determined
^{a}Ft. LVS challenge dose was 4.0 × cfu
^{b}Ft. LVSΩwbtA challenge dose was 4.0 × cfu

### Example 5

### Ft. LVSΩwbtA is Severely Defective in its Ability to Disseminate

### Materials and Methods

Dissemination studies. The Ft. LVS and Ft. LVSΩwbtA burdens in the spleens, lungs, kidneys, and livers were determined 4 days following challenge with 10⁷ cfu (i.d.) and 10⁴ cfu (i.n.), respectively. To determine bacterial load, the organs were harvested utilizing sterile technique, weighed, and then hand mashed prior to homogenization using a Stomacher® paddle action blender (Seward Ltd., Worthington WT1 09G). Serial dilutions were performed using sterile 1× PBS supplemented with 2% fetal bovine serum (FBS). 10 μl of each dilution were streaked on CHAR plates and incubated at 37° C. in the presence of 5% CO₂ until colonies were observed.

### Results

The mice challenged i.d. with the parent strain showed obvious signs of discomfort, and two of the five mice in this group died. The Ft. LVSΩwbtA-challenged mice did not show any signs of discomfort, and all the mice in both the i.d.- and i.n.-challenged mice of this group survived.

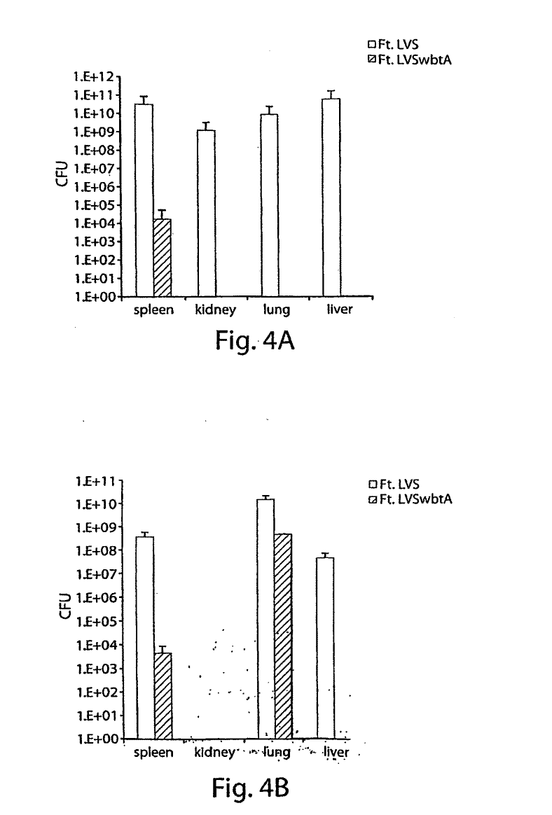

The ability of Ft. LVSΩwbtA and the parent strain to disseminate to the reticuloendothelial organs following challenge via the i.d. and i.n. route was examined. A 10-fold LD₅₀ (10⁷ cfu for i.d. and 10⁴ cfu for i.n.), as determined previously for F. tularensis LVS strain, was used in these experiments and organ load was determined at day four post-challenge. Analysis of the i.d.-challenged F. tularensis LVS mice indicated at least a two- to four-log increase of the cfu in the reticuloendothelial organs (FIG. 4A). Bacterial counts in spleens of mice challenged intradermally with the parent strain were ˜6.5 logs higher than those in spleens of Ft. LVSΩwbtA-challenged mice. (P value=0.0024). No detectable cfu were observed in the kidneys, lungs, or livers of the i.d.-challenged Ft. LVSΩwbtA mice. In contrast, mice challenged intradermally with the parent strain had at least 8-10 log cfu of bacteria/gram in these organs. Analysis of the organ loads from the i.n.-challenged mice indicated comparable organ load of the mutant and the parent strain in the lung tissue. However, the mutant was shown to be significantly restricted in its ability to disseminate to the reticuloendothelial tissues (FIG. 4B).

To investigate the basis for this severe dissemination defect, in vitro bactericidal assays were performed examining the mutant's sensitivity to complement-mediated killing. The wbtA mutant and the parent LVS strain were individually incubated with 20% heat-inactivated normal rabbit serum in the presence or absence of exogenous complement. The sensitivity of each strain to complement was determined by plating appropriate dilutions of each bacterium onto CHAH plates before and after 30 minutes of incubation. The wild-type LVS strain was fully resistant to killing, while 100% complement-dependent killing was documented for mutant strain Ft. LVSΩwbtA.

The degree of attenuation of the Ft. LVSΩwbtA strain was also evaluated by histopathological assessment of the liver and splenic tissue. Liver tissue from mice challenged with Ft. LVS showed obvious signs of inflammation with multiple foci of neutrophilic infiltration. In stark contrast, the liver tissue from Ft. LVSΩwbtA-challenged mice revealed only minor sporadic infiltration consistent with normal liver histology. Splenic histopathology at day 4 showed massive infiltration of neutrophils and macrophages (pyogranuloma) in Ft. LYS-challenged mice. Normal splenic architecture existed in splenic tissues from Ft. LVSΩwbtA-challenged mice. Similar results were also observed when an i.d. challenge dose of <10⁸ cfu of the Ft. LVSΩwbtA strain was administered.

### Example 6

### Immunization with Ft. LVSΩwbtA Confers Protective Immunity Against Subsequent Challenge with Lethal Doses of Both LVS and Wild-Type Virulent Type B Strains

### Materials and Methods

Protection studies. As described in Example 4, all mice with the exception of one mouse survived the challenge with F. tularensis LVS wbtA mutant strain. As a continuation of the studies in Example 4, the protective effect of the wbtA mutant was determined. In one experiment all the wbtA-immunized mice from Example 4 were challenged at day 28 with a 25-fold LD₅₀ of Ft. LVS via homologous or heterologous routes, i.e., intradermal and intranasal routes. In a second experiment naïve mice, mice immunized with Ft. LVS, and mice immunized with Ft. LVSΩwbtA were administered a lethal intradermal challenge of 17 cfu of the fully virulent FSC 108 type B strain of F. tularensis.

### Results

The results of the first experiment in this example are summarized in Table 4 (intranasal immunization) and Table 5 (intradermal immunization). Mice immunized intranasally were fully protected with an immunization dose of 10⁴ cfu (intranasal challenge, n=4) and 10⁵ cfu (intradermal challenge, n=4) (Table 4). Mice immunized intradermally were fully protected with an immunization dose of 10⁶ cfu (intranasal challenge, n=4) and 10⁴ cfu (intradermal challenge, n=4) (Table 5).

TABLE 4
Protection of male BALB/c mice after challenge with
approximately 25 fold LD₅₀ of F. tularensis LVS
strain, 28 days after intranasal immunization of mice with
increasing doses of F. tularensis LVSΩwbtA mutant.

| Mutant | Survival Ratio | | MTD | |
|---|---|---|---|---|
| Immunization | i.d. | i.n. | i.d. | i.n. |
| Dose (cfu) | Challenge | Challenge | Challenge | Challenge |

| | | | | |
|---|---|---|---|---|
| 10¹ | 2/4 | 0/4 | 4 | 11 |
| 10² | 2/4 | 0/4 | 4 | 13 |
| 10³ | 1/4 | 1/4 | 5 | 13 |
| 10⁴ | 3/4 | 4/4 | 4 | >21 |
| 10⁵ | 4/4 | 4/4 | >21 | >21 |
| 10⁶ | 4/4 | 4/4 | >21 | >21 |
| 10⁷ | 4/4 | 4/4 | >21 | >21 |

MTD, mean time to death in days
i.d challenge dose: 25 fold above the i.d. LD₅₀
i.n challenge dose: 25 fold above the i.n. LD₅₀
MTD of naïve BALB/c mice @ i.d. 10⁷: 4.5 days
MTD of naïve BALB/c mice @ i.n. 10⁴: 8.25 days

TABLE 5
Protection of male BALB/c mice after challenge with approximately
25 fold LD₅₀ of F. tularensis LVS strain, 28 days after
intradermal immunization of mice with increasing doses of
F. tularensis LVSΩwbtA mutant.

| | | |
|---|---|---|
| Bacterial | Survival Ratio | MTD |

| Inoculum | i.d. | i.n. | i.d. | i.n. |
|---|---|---|---|---|
| (cfu) | Challenge | Challenge | Challenge | Challenge |
| 10³ | 1/4 | 0/4 | 3.6 | 9.5 |
| 10⁴ | 4/4 | 3/4 | >21 | 13 |
| 10⁵ | 4/4 | 3/4 | >21 | 4 |
| 10⁶ | 4/4 | 4/4 | >21 | >21 |
| 10⁷ | 4/4 | 4/4 | >21 | >21 |
| 10⁸ | 4/4 | 4/4 | >21 | >21 |

MTD, mean time to death in days
i.d challenge dose: 25 fold above the i.d. LD₅₀
i.n challenge dose: 25 fold above the i.n. LD₅₀
MTD of naïve BALB/c mice @ i.d. 10⁷: 4.5 days
MTD of naïve BALB/c mice @ i.n. 10⁴: 8.25 days

These data demonstrate that, while this strain is avirulent in mice in comparison to the existing live vaccine strain, it yields very good protection against the development of experimental tularemia. This makes it a vastly superior strain for human use since the existing live vaccine strain is associated with side effects and safety concerns that have precluded its approval for use in humans.

Having demonstrated that immunization with Ft. LVSΩwbtA protects against lethal LVS challenge, it was also determined that immunization with Ft. LVSΩwbtA protected against challenge with a wild-type virulent type B strain. Mice immunized with Ft. LVS were used as controls in this experiment. As expected, mice immunized with Ft. LVS showed overt signs of infection (pilo-erection) between the fourth and seventh day post-vaccination and developed an obvious necrotic lesion at the site of inoculation. Additionally, in keeping with previous experience, some of the Ft. LVS-immunized mice died during the first week following of immunization with only 10³ bacterial cells. In stark contrast, mice immunized with 100,000-fold greater doses of with Ft. LVSΩwbtA strain showed no overt signs of illness. There were no deaths in this latter group, although they did develop transient dermal necrosis at the injection site.

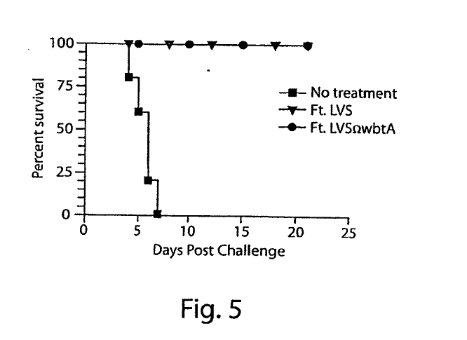

As shown in FIG. 5, following a lethal intradermal challenge of 17 cfu with the fully virulent FSC 108 type B strain of F. tularensis, all naïve mice but not Ft. LVS-immunized mice succumbed. Notably, when the same challenge was administered to mice immunized with the with Ft. LVSΩwbtA strain, although these mice showed symptoms of infection during the initial stages (between days 4-12 of infection), all the mice fully recovered and survived the lethal virulent type B challenge.

The studies presented in this example demonstrate that, despite the mutant's severely attenuated status, strain with Ft. LVSΩwbtA remains immunogenic, inducing immunity that effectively protects mice against lethal challenge with either LVS or a fully virulent type B clinical isolate of F. tularensis.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

We claim:

1. A vaccine composition comprising an effective amount of a live attenuated avirulent strain of Francisella tularensis, wherein the live attenuated avirulent strain has reduced expression of wild-type O antigen as compared to pathogenic Francisella tularensis, and a pharmaceutically acceptable carrier.

2. The vaccine composition of claim 1, wherein the reduced expression of wild-type O antigen is no expression of wild-type O antigen.

3. The vaccine composition of claim 1, wherein the effective amount is at least 10⁴ colony forming units.

4. The vaccine composition of claim 1, wherein the effective amount is 10⁴ to 10⁸ colony forming units.

5. The vaccine composition of claim 1, wherein the reduced expression results from a mutation of a gene affecting O-antigen biosynthesis.

6. The vaccine composition of claim 5, wherein the mutation is a mutation of a gene within an O-antigen biosynthesis locus.

7. The vaccine composition of claim 6, wherein the mutation is a mutation of wbtA.

8. A method of vaccinating a mammal against a pathogenic strain of Francisella tularensis, comprising administering to the mammal an effective amount of a live attenuated avirulent strain of Francisella tularensis, wherein the live attenuated avirulent strain has reduced expression of wild-type O antigen as compared to pathogenic Francisella tularensis.

9. The method of claim 8, wherein the reduced expression of wild-type O antigen is no expression of wild-type O antigen.

10. The method of claim 8, wherein the effective amount is at least 10⁴ colony forming units.

11. The method of claim 8, wherein the effective amount is 10⁴ to 10⁸ colony forming units.

12. The method of claim 8, wherein the reduced expression results from a mutation of a gene affecting O-antigen biosynthesis.

13. The method of claim 12, wherein the mutation is a mutation of a gene within an O-antigen biosynthesis locus.

14. The method of claim 13, wherein the mutation is a mutation of wbtA.

15. The method of claim 8, wherein the administering comprises administering intranasally.

16. The method of claim 8, wherein the administering comprises administering intradermally.

17. The method of claim 8, wherein the mammal is a human.

18. A method of making a live attenuated avirulent strain of Francisella tularensis, comprising introducing a mutation into a gene within an O-antigen biosynthesis locus of a pathogenic strain of Francisella tularensis, wherein the mutation results in reduced expression of wild-type O antigen by the pathogenic strain of Francisella tularensis.

19. The method of claim 18, wherein the gene is wbtA.

20. The method of claim 18, wherein the pathogenic strain of Francisella tularensis is Francisella tularensis type A.

21. A method for identifying a candidate vaccine of a live attenuated avirulent strain of Francisella tularensis, comprising
comparing expression of wild-type O antigen by a mutant strain of Francisella tularensis to expression of wild-type O antigen by a pathogenic strain of Francisella tularensis; and
identifying the mutant strain of Francisella tularensis as a candidate vaccine when the mutant strain has reduced expression of wild-type O antigen compared to the pathogenic strain.

22. The method of claim 21, wherein the reduced expression of wild-type O antigen compared to the pathogenic strain is no expression of wild-type O antigen.

23. The method of claim 21, wherein the reduced expression results from a mutation of a gene affecting O-antigen biosynthesis.

24. The method of claim 23, wherein the mutation is a mutation of a gene within an O-antigen biosynthesis locus.

25. The method of claim 24, wherein the mutation is a mutation of wbtA.

26. A method for identifying an agent that binds to and inhibits activity of an enzyme involved in biosynthesis of wild-type O antigen of a pathogenic strain of Francisella tularensis, the method comprising
contacting an enzyme involved in biosynthesis of wild-type O antigen of a pathogenic strain of Francisella tularensis with a test agent;
measuring activity of the enzyme in presence of the test agent; and
determining whether the test agent binds to and inhibits activity of the enzyme.

27. The method of claim 26, wherein the enzyme involved in biosynthesis of wild-type O antigen of the pathogenic strain of Francisella tularensis is WbtA.

28. A method for identifying an agent that binds to and inhibits activity of a gene involved in biosynthesis of wild-type O antigen of a pathogenic strain of Francisella tularensis, the method comprising
contacting a gene involved in biosynthesis of wild-type O antigen of a pathogenic strain of Francisella tularensis with a test agent;
measuring activity of the gene in presence of the test agent; and
determining whether the test agent binds to and inhibits activity of the gene.

29. The method of claim 28, wherein the gene involved in biosynthesis of wild-type O antigen of the pathogenic strain of Francisella tularensis is wbtA.


SEQUENCE LISTING (8 sequences)

SEQ ID NO: 1 - DNA, 1737 bp, Francisella tularensis
atgtctttctacgataatagaacgcttaatttcgtggtaataatagttttaactattattactgttaattggactttctatattttcaagcaagatgttaatttacattttttacttgcattagttttgctgagatgcttgtcatcttttttactacttagagattatatggctagttggcgtaagtcgactcaaaaaacttttttacgtaaggcttttattaatttgccagtatttttcatagtggcattatttttttatggcaaagtcactttttcgttgatattctctgagtttttattttatgtttttttgatcagtttaagtgtctacttttattggtatttgatgaacagaggatcagtggataaaagtaaaactgcggttatttatggtgcaggtgctgcaggaacaaagattgctcaagaacttgcttctgctggttatcgcatcaaatgttttgttgatgacaatgaaactttacaaaaaagaagtattgatagtaaaaaggttctatctaaagctgaattaacaaaactattgctatctagtagatttgaccttttggttattgcattgccaagaaatgcaaaccaagtagtcaaaaatatatataaagaatttgaaaaggattttaatcagattagaattatgccgcctcttgaggaaattcttcaagatgagaattttatgtcacagttgaagcctgtttcactctatgatctattagcgcgtgatactaagagtttagataaagaatctatctctaattttatcaaaaataaggtggtgctagtcacaggagctggaggtagtataggttctgaaatagtacatcaatgtatcaagtatcaggcaaaagagttgatattggttgatcatagtgagtttaacttatataaaattactgaggagtgtagtcattttaatatcaatagtgtgctatgttctgtttgtgatagaaaagcattggctgaggtttttcaaaagtatactccaaatatagtatttcatgctgctgcctacaagcatgttcccttagttgaggagaatatctctagagcaattagaaataatatcttaggtactaagaatgctatagatctggctatagaagctggtgttgagtcatttatattgatttccactgataaagcagtgcgaccaacgaatgttatgggggctaccaagagagtttgtgagctgtatttacagaatgttgatcccaaaaataccaagcttgctgcagtgcgttttggtaatgtgcttggtagtagtggcagtgtgattccaaaatttgaagagcaaataagaaaaggtggtcctgttacagttactcatcctgaaattacacgttattttatgttgataccagaagcttgtgaactggtcctacaagctggtgctattgcaaaaaattcagaggtctttgtcttagatatggggcaacctgtcaagattattgatcttgctaaacaatttattagactttctggtagaggtgatattgatattaaaatagttggtttgcgtccaggagagaaactttacgaagagcttttgatagaggaagatgatgttagtaccgactataaagatatttttattggtagaaggactttttacgatattaatactctaaaccaagatattgaatcgttgatcaaggatgatgttgatcagcttgtgatattaaagaaaattgttccggaatttgaacatagattgaatgggtag

SEQ ID NO: 2 - protein, 578 aa, Francisella tularensis
MSFYDNRTLNFVVIIVLTIITVNWTFYIFKQDVNLHFLLALVLLRCLSSFLLLRDYMASWRKSTQKTFLRKAFINLPVFFIVALFFYGKVTFSLIFSEFLFYVFLISLSVYFYWYLMNRGSVDKSKTAVIYGAGAAGTKIAQELASAGYRIKCFVDDNETLQKRSIDSKKVLSKAELTKLLLSSRFDLLVIALPRNANQVVKNIYKEFEKDFNQIRIMPPLEEILQDENFMSQLKPVSLYDLLARDTKSLDKESISNFIKNKVVLVTGAGGSIGSEIVHQCIKYQAKELILVDHSEFNLYKITEECSHFNINSVLCSVCDRKALAEVFQKYTPNIVFHAAAYKHVPLVEENISRAIRNNILGTKNAIDLAIEAGVESFILISTDKAVRPTNVMGATKRVCELYLQNVDPKNTKLAAVRFGNVLGSSGSVIPKFEEQIRKGGPVTVTHPEITRYFMLIPEACELVLQAGAIAKNSEVFVLDMGQPVKIIDLAKQFIRLSGRGDIDIKIVGLRPGEKLYEELLIEEDDVSTDYKDIFIGRRTFYDINTLNQDIESLIKDDVDQLVILKKIVPEFEHRLNG

SEQ ID NO: 3 - DNA, 32 bp, Artificial sequence
  Synthetic oligonucleotide
tctagcggccgcactatacccttcaagctttg

SEQ ID NO: 4 - DNA, 35 bp, Artificial sequence
  Synthetic oligonucleotide
cgttgaatatggctcataacaatcttactcctttg

SEQ ID NO: 5 - DNA, 32 bp, Artificial sequence
  Synthetic oligonucleotide
tctagcggccgcttgaaaattttttttttgac

SEQ ID NO: 6 - DNA, 32 bp, Artificial sequence
  Synthetic oligonucleotide
ggagtaagattgttatgagccatattcaacgg

SEQ ID NO: 7 - DNA, 30 bp, Artificial sequence
  Synthetic oligonucleotide
gtacggattccaaccctgaagcttgcttgc

SEQ ID NO: 8 - DNA, 21 bp, Artificial sequence
  Synthetic oligonucleotide
gattccggataacaggttggc